(19) United States
(12) Patent Application Publication
(10) Pub. No.: US 2026/0161349 A1
(43) Pub. Date: Jun. 11, 2026

(54) INTEGRATED ASSISTANCE SYSTEMS AND METHODS

(71) Applicant: Simon Leonardo LIU, Troy, MI (US)

(72) Inventor: Simon Leonardo LIU, Troy, MI (US)

(21) Appl. No.: 18/969,264

(22) Filed: Dec. 5, 2024

(51) Int. Cl. G06F 3/16; A61B 5/00; A61B 5/256; A61B 5/291; A61B 5/374; A61B 5/375; G06F 3/01; G06V 10/70; G10L 13/033

(52) U.S. Cl. CPC G06F 3/167; A61B 5/0006; A61B 5/0022; A61B 5/256; A61B 5/291; A61B 5/374; A61B 5/375; A61B 5/4088; A61B 5/741; G06F 3/015; G06V 10/70; G10L 13/033

(57) ABSTRACT

An integrated assistance system and method are provided. The integrated assistance system includes: a wearable device to obtain electroencephalogram (EEG) signals from a user through one or more electrode channels; a visual sensing module to capture the environment image in front of the user; a unit to analyze the environment image content; an analysis unit to determine a cognitive load index (CLI) and an asymmetry index (ASY) using EEG signals; and a unit to determine the user's brain state, including recognition based on CLI and memory state based on ASY. The integrated assistance system provides support by analyzing the user's brain activity and surroundings through visual recognition technology, ultimately offering speech assistance to the user, especially for MCI patients.

### TECHNICAL FIELD

The present disclosure relates to the field of cognitive assistance technologies, and in particular, to an integrated assistance system and method.

### BACKGROUND

As the global population continues to age, there is a growing need for innovative solutions to support elderly individuals, particularly those suffering from mild cognitive impairment (MCI). Individuals with MCI often struggle with recognizing and remembering people, objects, and environments, significantly impacting their daily lives and reducing their ability to live independently. The care required for these individuals demands substantial resources, including time, money, and patience, from caregivers and healthcare systems.

Traditional service robots designed to assist the elderly have primarily focused on physical tasks, such as aiding in mobility or performing routine activities. However, these robots often lack the cognitive and emotional intelligence necessary to address the complex needs of MCI patients. The integration of advanced technologies, including environmental perception, intention recognition, and compliance control, is crucial for the development of effective elderly care systems that can provide both physical and cognitive support.

Recent advancements in human-robot interaction (HRI) have demonstrated the potential of robots to seamlessly integrate into human life, particularly with the developments in Industry 4.0. Robots are now capable of flexible interaction within various environments, including hospitals, homes, and care facilities. This adaptability suggests that robots can evolve beyond simple task execution to become integral partners in elder care, addressing both physical and cognitive demands.

However, current systems still face significant limitations. Many existing solutions are costly, lack the necessary adaptability, and fail to provide the cognitive support required for MCI patients. The incorporation of electroencephalogram (EEG) signals, combined with advanced visual sensing technologies and artificial intelligence (AI), represents a promising approach to overcoming these challenges. By analyzing EEG data alongside environmental inputs, it becomes possible to tailor assistance to the specific cognitive and emotional states of the user, thereby enhancing both the efficacy and personalization of care.

Based on this, it is necessary to provide an integrated assistance system and method, which may address the shortcomings of current elderly care robots, offering a more holistic and cost-effective solution that enhances the overall quality of life for individuals with MCI.

### SUMMARY

The present disclosure provides an integrated assistance system designed to enhance the quality of life for individuals with mild cognitive impairment (MCI) by combining visual sensing, electroencephalogram (EEG) signal analysis, and advanced artificial intelligence (AI) algorithms.

Embodiments of the present disclosure may provide an integrated assistance system, comprising: a wearable device configured to obtain one or more electroencephalogram (EEG) signals of a user through one or more electrode channels; an electroencephalogram (EEG) signal sensing module configured to obtain cognitive information by monitoring the one or more EEG signals of the user, the cognitive information including a cognitive load and a memory state, wherein the EEG signal sensing module further includes; a preprocessing unit configured to preprocess the one or more EEG signals; an analysis unit configured to determine a cognitive load index (CLI) and an asymmetry index (ASY) by determining a power spectral density (PSD) across a plurality of frequency bands based on the preprocessed EEG signals; and a cognitive identification unit configured to determine the cognitive load based on the CLI and determine the memory state based on the ASY.

Embodiments of the present disclosure may provide an integrated assistance method, comprising: obtaining one or more electroencephalogram (EEG) signals of a user through one or more electrode channels; obtaining cognitive information by monitoring the one or more EEG signals of the user, the cognitive information including a cognitive load and a memory state, wherein the obtaining cognitive information by monitoring the one or more EEG signals of the user includes: preprocessing the one or more EEG signals; determining a cognitive load index (CLI) and an asymmetry index (ASY) by determining a power spectral density (PSD) across a plurality of frequency bands based on the preprocessed EEG signals; and determining the cognitive load based on the CLI and determine the memory state based on the ASY.

Embodiments of the present disclosure may provide a non-transitory computer-readable storage medium storing computer instructions, wherein when reading the computer instructions in the storage medium, a computer executes the integrated assistance method.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an exemplary architecture of an integrated assistance system according to some embodiments of the present disclosure;

FIG. 2 is a flowchart illustrating an exemplary operation process of an integrated assistance system according to some embodiments of the present disclosure;

FIG. 3 is a schematic diagram illustrating an exemplary integrated assistance system according to some embodiments of the present disclosure;

FIG. 4 is a block diagram illustrating an exemplary integrated assistance system according to some embodiments of the present disclosure; and

FIG. 5 is a flowchart illustrating an exemplary integrated assistance method according to some embodiments of the present disclosure.

### DETAILED DESCRIPTION

In the following detailed description, numerous specific details are set forth by way of examples in order to provide a thorough understanding of the relevant disclosure. Obviously, drawings described below are only some examples or embodiments of the present disclosure. Those skilled in the art, without further creative efforts, may apply the present disclosure to other similar scenarios according to these drawings. It should be understood that the purposes of these illustrated embodiments are only provided to those skilled in the art to practice the application, and not intended to limit the scope of the present disclosure. Unless obviously obtained from the context or the context illustrates otherwise, the same numeral in the drawings refers to the same structure or operation.

It will be understood that the terms “system,” “unit,” and/or “module” used herein are one method to distinguish different components, elements, parts, sections, or assemblies of different levels in ascending order. However, the terms may be displaced by other expressions if they may achieve the same purpose.

The terminology used herein is for the purposes of describing particular examples and embodiments only and is not intended to be limiting. As used herein, the singular forms “a,” “an,” and “the” may be intended to include the plural forms as well, unless the context clearly indicates otherwise. It will be further understood that the terms “include” and/or “comprise,” when used in this disclosure, specify the presence of integers, devices, behaviors, stated features, steps, elements, operations, and/or components, but do not exclude the presence or addition of one or more other integers, devices, behaviors, features, steps, elements, operations, components, and/or groups thereof.

The flowcharts used in the present disclosure illustrate operations that systems implement according to some embodiments of the present disclosure. It is to be expressly understood, the operations of the flowcharts may be implemented not in order. Conversely, the operations may be implemented in an inverted order, or simultaneously. Moreover, one or more other operations may be added to the flowcharts. One or more operations may be removed from the flowcharts.

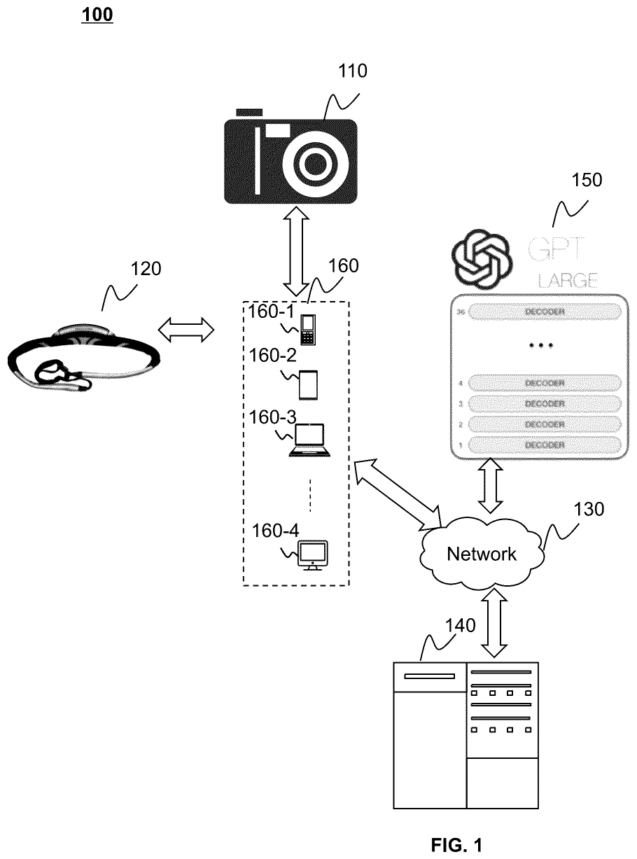

FIG. 1 is a schematic diagram illustrating an exemplary architecture of an integrated assistance system according to some embodiments of the present disclosure.

As shown in FIG. 1, the integrated assistance system 100 may include a visual sensing module 110, a wearable device 120, a network 130, a processing device 140, a personal chat robot 150, and at least one terminal device 160.

The visual sensing module 110 is configured to obtain environment information from an environment of a user (e.g., an MCI patient, an elder, etc.). In some embodiments, the visual sensing module 110 is equipped with image sensors that capture environment images from the environment of the user.

The wearable device 120 is a device used to monitor a physiological state (e.g., a physiological signal) of a user. The wearable device 120 may include vests, watches, head-mounted helmets, smart glasses, etc., that the user can wear comfortably. In some embodiments, the wearable device 120 is configured to obtain one or more electroencephalogram (EEG) signals of the user through one or more electrode channels. The one or more electrode channels are channels for collecting the EEG signals, and may include an AF7 channel, an AF8 channel, a TP9 channel, and a TP10 channel. The AF7 channel is located on the left forehead and is configured to detect EEG signals in the left forehead area. The AF8 channel is located on the right forehead and is configured to detect EEG signals in the right forehead area. The TP9 channel is located on the left temporal lobe and is configured to detect EEG signals in the left temporal lobe area. The TP10 channel is located on the right temporal lobe and is configured to detect EEG signals in the right temporal lobe area. In some embodiments, each electrode channel corresponds to a plurality of frequency bands, including a δ band (in a range of 0.5-4 Hz), a θ band (in a range of 4-8 Hz), a α band (in a range of 8-14 Hz), a β band (in a range of 14-30 Hz), and a γ band (in a range of 30-45 Hz).

The network 130 may be or may include a public network (e.g., the Internet), a private network (e.g., a local area network (LAN), a wide area network (WAN), etc.), a wired network, a wireless network, etc. The network 130 may include any suitable network that facilitates the exchange of information and/or data of the integrated assistance system. In some embodiments, one or more components (e.g., the visual sensing module 110, the wearable device 120, the processing device 140, and the personal chat robot 150) of the integrated assistance system 100 may exchange information and/or data with each other via the network 130. For example, the processing device 140 may obtain the environment information from the visual sensing module 110 via the network 130. As another example, the processing device 140 may obtain the EEG signals from the wearable device 120 via the network 130.

The processing device 140 may process the data and/or information obtained from the visual sensing module 110 and the wearable device 120. For example, the processing device 140 may obtain cognitive information by monitoring the one or more EEG signals. As another example, the processing device 140 may generate fusion information based on the environment information and the cognitive information.

The personal chat robot 150 refers to a robot that provides real-time, context-aware assistance through TTS (Text-to-Speech) with a family-style voice, enhancing user comfort and engagement. In some embodiments, the personal chat robot 150 is powered by advanced AI algorithms. For example, the personal chat robot 150 may include cloud-based ChatGPT, etc.

The at least one terminal device 160 may communicate and/or connect with the processing device, 10, a personal chat robot 150, etc., through the network 130. For example, the at least one terminal device 160 may send one or more control commands to the personal chat robot 150 to control the personal chat robot 150 to provides assistance for the user. In some embodiments, the at least one terminal device 160 may include one of a mobile device 160-1, a tablet 160-2, a laptop 160-3, a desktop computer 160-4, or other devices with input and/or output functions, or a combination thereof.

In some embodiments, the wearable device 120 may integrate one or more components (e.g., the visual sensing module 110) in the integrated assistance system, allowing for continuous monitoring and analysis of the EEG signals and/or the environment information. In some embodiments, the processing device 140 may be an independent component or may be integrated in other components in the integrated assistance system.

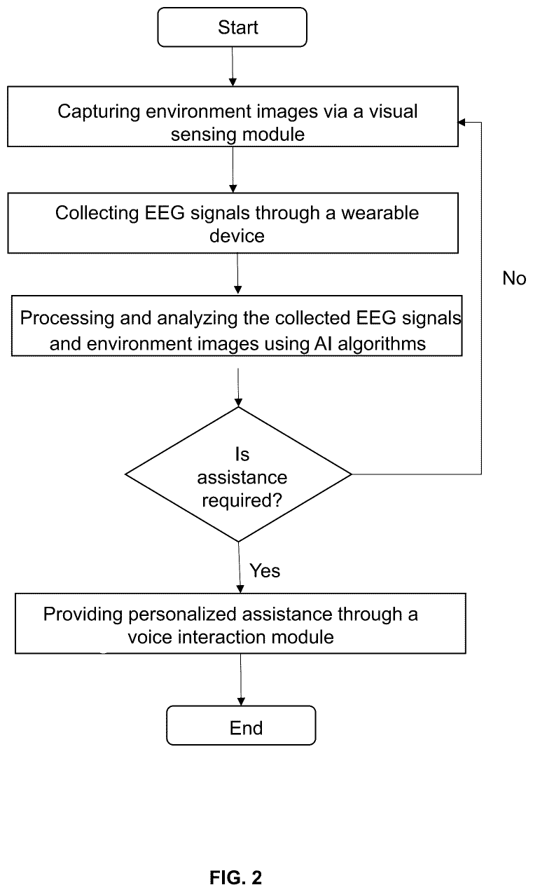

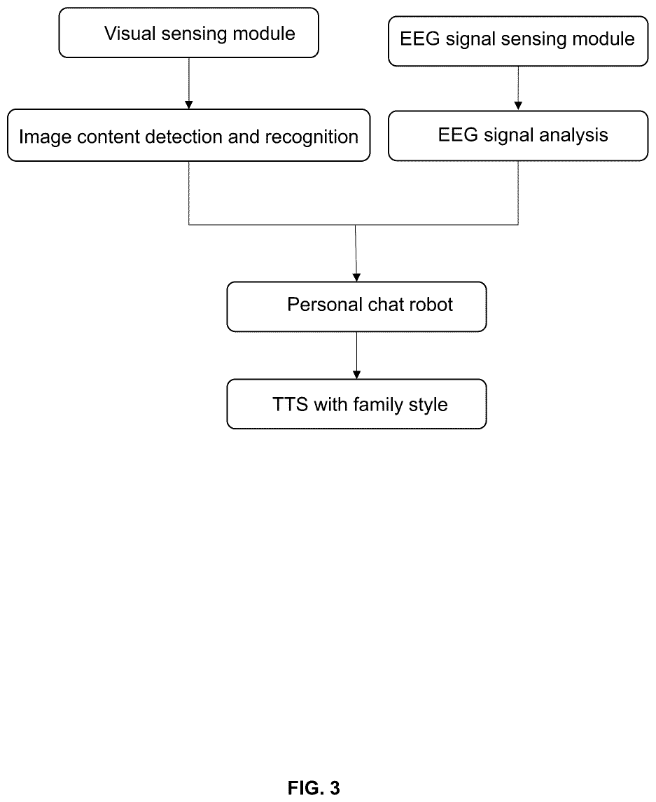

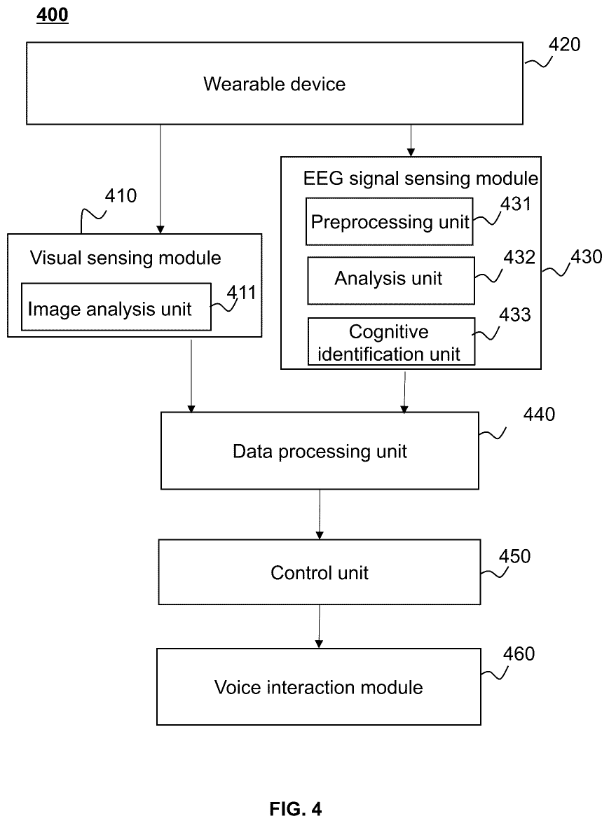

FIG. 2 is a flowchart illustrating an exemplary operation process of an integrated assistance system according to some embodiments of the present disclosure. FIG. 3 is a schematic diagram illustrating an exemplary integrated assistance system according to some embodiments of the present disclosure. FIG. 4 is a block diagram illustrating an exemplary integrated assistance system according to some embodiments of the present disclosure.

As shown in FIG. 4, an integrated assistance system 400 may include a visual sensing module 410, a wearable device 420, an electroencephalogram (EEG) signal sensing module 430, a data processing unit 440, a control unit 450, and a voice interaction module 460.

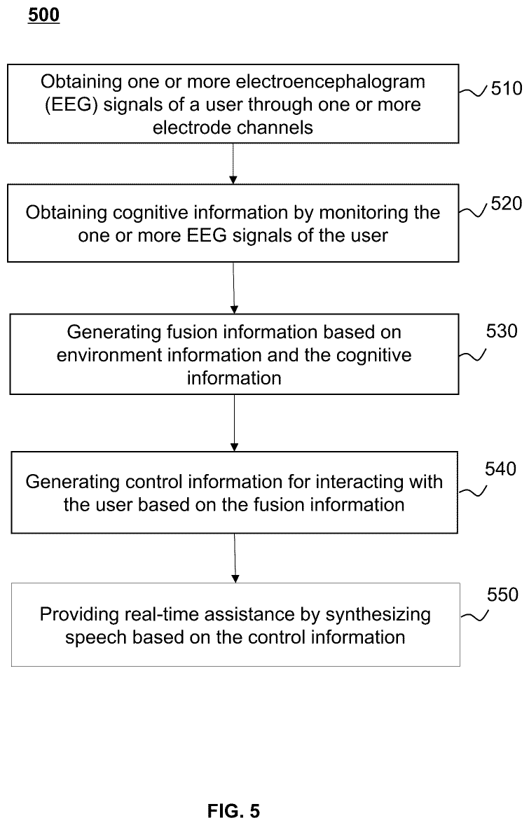

The visual sensing module 410 is configured to obtain environment information from an environment of a user. The environment information refers to scene information from the environment of the user. For example, the environment information may include information related to buildings, objects, people, etc., in the environment. In some embodiments, the visual sensing module 410 includes an analysis unit 411, and the analysis unit 411 411 is further configured to determine a target object from the environment of the user using an image recognition model, the image recognition model being a machine learning model. The target object refers to an item or people in the environment of the user. In some embodiments, a captured environment image, which is in front of the user, may be processed by the analysis unit 411 in the visual sensing module 410. The analysis unit 411 uses deep learning-based algorithms to analyze the content of the environment image, which can then be understood through the image. For more descriptions regarding a process for obtaining the environment information and determining the target object may be found in FIG. 5 and related descriptions thereof.

The wearable device 420 is a device used to monitor a physiological state (e.g., a physiological signal) of a user. The structure and function of the wearable device 420 are similar to those of the wearable device 120, which will not be described in detail here.

In some embodiments of the present disclosure, the user's comfort is fully considered, and lightweight wearable devices are used, which can be easily operated by elderly users.

The electroencephalogram (EEG) signal sensing module 430 is configured to obtain cognitive information by monitoring the one or more EEG signals of the user. The cognitive information includes a cognitive load and a memory state. In some embodiments, the EEG signal sensing module 430 includes a preprocessing unit 431, an analysis unit 432, and a cognitive identification unit 433. The preprocessing unit 431 is configured to preprocess the one or more EEG signals. The analysis unit 432 is configured to determine a cognitive load index (CLI) and an asymmetry index (ASY) by determining a power spectral density (PSD) across a plurality of frequency bands based on the preprocessed EEG signals. The cognitive identification unit 433 is configured to determine the cognitive load based on the CLI and determine the memory state based on the ASY. For detailed descriptions regarding the cognitive load, the memory state, the CLI, the ASY, and the PSD may be found in FIG. 5 and related descriptions thereof.

The data processing unit 440 is configured to generate fusion information based on environment information and the cognitive information. For detailed descriptions regarding the fusion information may be found in FIG. 5 and related descriptions thereof.

The control unit 450 is configured to generate control information for interacting with the user based on the fusion information. In some embodiments, the control unit 450 is configured to generate the control information for interacting with the user in a user-initiated mode or a cloud-initiated mode. For detailed descriptions regarding the control information, the user-initiated mode, and the cloud-initiated mode may be found in FIG. 5 and related descriptions thereof.

The voice interaction module 460 is configured to provide real-time assistance by synthesizing speech based on the control information. In some embodiments, the voice interaction module includes a text-to-speech (TTS) engine configured to synthesize the speech using a voice style transfer technique. In some embodiments, the voice interaction module 460 may include or implemented as a personal chat robot. For detailed descriptions regarding the real-time assistance and speech synthesis may be found in FIG. 5 and related descriptions thereof.

As shown in FIG. 2 and FIG. 4, the operation process of the integrated assistance system includes: capturing environment images via the visual sensing module 410, and collecting EEG signals through the wearable device 420. The collected EEG signals are further processed and analyzed using AI algorithms to determine whether assistance is required. If assistance is required, personalized assistance is provided through the voice interaction module 460.

As shown in FIG. 3 and FIG. 4, the visual sensing module 410 obtains environment information which is processed and analyzed along with EEG signals from the wearable device 420 to derive meaningful insights (e.g., the fusion information). For example, an image content detection and recognition operation is performed on the captured environment image (e.g., by the image analysis unit 411), an EEG data analysis operation is performed on the EEG signals (e.g., by the EEG signal sensing module 430) to obtain the environment information and the cognitive information, and the fusion information is generated by the data processing unit 440 based on the environment information and the cognitive information. The integrated assistance system further uses the processed data (i.e., the fusion information) to interact with the user through a personal chat robot, offering real-time assistance. For example, the control unit 450 is configured to generate control information for interacting with the user based on the fusion information. Further, the voice interaction module 460 is configured to provide real-time assistance by synthesizing speech based on the control information. In some embodiments, the voice interaction module 460 is configured to provide the real-time assistance through the personal chat robot. In some embodiments, the personal chat robot is regarded as an implementation of the voice interaction module 460 In some embodiments, the interaction between the integrated assistance system and the user is further enhanced by TTS with a family-style voice, providing a familiar and comforting user experience for MCI patients. For example, the voice interaction module 460 (or the personal chat robot) includes a TTS engine configured to synthesize the speech using a voice style transfer technique, and the speech mimics voice of a specific person (e.g., a family member).

In some embodiments of the present disclosure, by combining real-time EEG data analysis, visual perception, and personalized voice interaction, the system is able to provide tailored guidance and support to meet the cognitive and emotional needs of MCI patients.

FIG. 5 is a flowchart illustrating an exemplary integrated assistance method according to some embodiments of the present disclosure. As shown in FIG. 5, a process 500 may include the following operations. In some embodiments, the process 500 may be executed by a processor in one or more components (e.g., the visual sensing module 410, the wearable device 420, the EEG signal sensing module 430, the data processing unit 440, the control unit 450, and the voice interaction module 460) of an integrated assistance system. In some embodiments, one or more components (e.g., the visual sensing module 410, the EEG signal sensing module 430, the data processing unit 440, the control unit 450, and the voice interaction module 460) of the integrated assistance system are integrated on a processor (or a processing device, e.g., the processing device 140) of the integrated assistance system, and the process 500 may be executed by the processor.

In 510, the processor (e.g., the wearable device 420) obtains one or more electroencephalogram (EEG) signals of a user through one or more electrode channels.

The EEG signals refer to signals collected by detecting a brain activity. The EEG signals are used for identifying cognitive and emotional states of the user, enabling the integrated assistance system to adapt the interactions with the user accordingly. In some embodiments, the processor may obtain the one or more EEG signals of the user through the one or more electrode channels disposed on a wearable device. For example, the processor may obtain the one or more EEG signals of the user through the one or more electrode channels in real-time or at a preset time interval (e.g., 1 s, 10 s, etc.). The preset time interval may be determined according to scenario requirements. In some embodiments, the processor may obtain EEG signals collected by at least one of the electrode channels. For example, the processor may obtain EEG signals collected by an AF7 channel and an AF8 channel. For more descriptions regarding the electrode channels may be found in FIG. 1 and related descriptions thereof.

In 520, the processor (e.g., the EEG signal sensing module 430) obtains cognitive information by monitoring the one or more EEG signals of the user.

The cognitive information reflects an ability to recognize things of the brain. The cognitive information may include a cognitive load and a memory state. The cognitive load is used to assess a cognitive ability of the user to understand things (e.g., a target object). For example, the cognitive load may be divided into no cognitive load, a moderate cognitive load; and a high cognitive load. The memory state refers to a state in which the user recall things (e.g., the target object). For example, the memory state may be divided into no significant memory recall, a mild memory recall; and an intense memory recall. For a definition of the target object may be found in FIG. 4, and for more descriptions of the target object may be found in operation 530.

In some embodiments, the cognitive load may be represented by a cognitive load index (CLI), and the memory state may be represented by an asymmetry index (ASY).

In some embodiments, the processor may preprocess the one or more EEG signals; determine the CLI and the ASY by determining a power spectral density (PSD) across a plurality of frequency bands based on the preprocessed EEG signals; and determine the cognitive load based on the CLI and determine the memory state based on the ASY.

In some embodiments, the one or more EEG signals may be processed using EEGLab software, which supports detailed analysis through spectral density graphs and topographical brain maps. The preprocessing includes performing at least downsampling, artifact removal, and filtering on raw EEG signals to isolate relevant frequency bands, and then performing independent component analysis (ICA) on the signals to separate neural sources from physiological artifacts. After the ICA is finished, the processor may perform a Fourier Transform (FT) on the preprocessed EEG signals to analyze a power distribution across the plurality of frequency bands, thereby obtaining the power spectral density (PSD) across the plurality of frequency bands. For descriptions of the frequency bands may be found in FIG. 1.

For example, the PSD is determined as follows:

$$P ( f ) = \frac{1}{N} {\vert {\sum }_{n = 0}^{N - 1} x ( n ) {e}^{- j 2 π f n / N} \vert}^{2} , \qquad ( 1 )$$

where P(f) denotes a power at a frequency f; x(n) denotes a time series signal; N is a total count of sampling points.

In some embodiments, the cognitive load index (CLI) is determined as follows:

$$CLI = \frac{{P}_{θ}}{{P}_{α}} , \qquad ( 2 )$$

where P_{θ} denotes a power of the one or more electrode channels in the θ band, which is usually associated with focused attention and complex cognitive tasks, and P_{α} denotes a power of the one or more electrode channels in the α band, which is usually associated with relaxation, memory load, and light cognitive load; wherein

$${P}_{θ} = {P}_{θ}^{A F 7} + {P}_{θ}^{A F 8} + {P}_{θ}^{T P 9} + {P}_{θ}^{T P 10} , and$$

$${P}_{α} = {P}_{α}^{A F 7} + {P}_{α}^{A F 8} + {P}_{α}^{T P 9} + {P}_{α}^{T P 10} .$$

In some embodiments, the processor may determine the cognitive load based on the CLI. For example, if the CLI is smaller than 1.5, it means there is no cognitive load; if the CLI is not less than 1.5 and not greater than 3, it means there is a moderate cognitive load; and if the CLI is greater than 3, it means there is a high cognitive load.

In some embodiments, the ASY is determined based on a following equation (2):

$$ASY = \frac{R {H}_{δ} - L {H}_{δ}}{{δ}_{A S Y}} , \qquad ( 3 )$$

where RH_{δ} denotes a power of a right hemisphere in the δ band, which is calculated from the powers of AF8 and TP10 channels in the δ band, i.e.,

$$R {H}_{δ} = {P}_{δ}^{A F 8} + {P}_{θ}^{TP 10} ,$$

ana LH_{δ} denotes a power of a left hemisphere in the δ band, which is calculated from the powers of AF7 and TP9 channels in the δ band, i.e.,

$$L {H}_{δ} = {P}_{δ}^{A F 7} + {P}_{θ}^{T P 9} ;$$

and δ_{ASY} denotes a standard deviation of an asymmetry index during a baseline period.

The asymmetry index is used to quantify asymmetry of the power of the δ band between the left and right hemispheres, which may be expressed by:

$${ASY}_{δ} = \frac{R {H}_{δ} - L {H}_{δ}}{R {H}_{δ} + L {H}_{δ}} , \qquad ( 4 )$$

and δ_{ASY} may be determined by:

$${δ}_{A S Y} = \frac{\vert {ASY}_{δ}^{m} - A S {Y}_{δ}^{r} \vert}{\vert {ASY}_{δ}^{r} \vert} , where {ASY}_{δ}^{m} \qquad ( 5 )$$

is an ASY in a memory task state; and

$${ASY}_{δ}^{r}$$

is an ASY in a resting state.

In some embodiments, the processor may determine the memory state based on the ASY. For example, if an absolute value of the ASY is not greater than 0.2, it means there is no significant memory recall; if the absolute value of the ASY is greater than 0.2 and not greater than 0.5, it means there is a mild memory recall; and if the absolute value of the ASY is greater than 0.5, it means there is an intense memory recall.

In 530, the processor (e.g., the data processing unit 440) generates fusion information based on environment information and the cognitive information.

In some embodiments, the processor may obtain the environment information from an environment of the user. For example, the processor may obtain environment images through image sensors such as cameras, monitoring devices, etc., in real-time or at a preset time interval (e.g., 1 s, 10 s, etc.), and then process the environment images to obtain the environment information using any available technology, such as OpenAI. The preset time interval may be determined according to scenario requirements.

In some embodiments, the processor may determine the target object from the environment of the user using an image recognition model, the image recognition model being a machine learning model. For example, the processor preprocesses the environment information using an edge computing technique, ensuring that the system may function in real-time, and detects and recognizes the target object using a YOLOv5 model, which excels in identifying and localizing objects within segmented regions. The YOLOv5 model is particularly effective for determining the target object due to its real-time detection capabilities and high accuracy in identifying objects even in complex scenes. In some embodiments, the processor may determine the target object from the environment of the user by segmenting and processing the environment images using an image segmentation model. The image segmentation model may be visual recognition AI model such as OpenAI, which can provide more accurate image analysis, for example, the image segmentation model includes a backbone feature extractor, an instance segmentation module, a non-maximum suppression module, etc. In some embodiments, the target object and attributes of the target object are transformed into text information using a transformer based on the visual sensing module, enabling the integrated assistance system to generate coherent context-aware interactions. The text information refers to semantic information used to describe the environment of the user, objects and people in the environment. For more descriptions of the text information may be found in operation 540.

The fusion information refers to information related to the target object. For example, the fusion information may include the name of the target object or a relationship thereto. In some embodiments, the processor is configured to generate the fusion information based on the environment information and the cognitive information through a plurality of ways. For example, the processor may preset a comparison table of the cognitive load, the memory state, and the environment information, then determine the target object from the environment of the user, and determine the fusion information of the target object from the environment information according to the cognitive load or the memory state by looking up the comparison table.

Based on the environment information and cognitive information, current fusion information of an MCI patient can be accurately judged based on brain activities and environment images, making it easier to provide personalized assistance to the patient based on the fusion information.

In 540, the processor (e.g., the control unit 450) generates control information for interacting with the user based on the fusion information.

The control information refers to information predicted based on fusion information and used to assist the user in cognition. For example, the control information may be dialogue information used to remind the user to identify the target object corresponding to the fusion information.

In some embodiments, the processor may generate the control information for interacting with the user based on the fusion information. For example, in response to the user not knowing clearly about the target object (for example, not remembering the name of the target object, etc.), the processor may generate the control information to introduce the target object to the user. In some embodiments, the processor inputs the text information of the target object and the fusion information into a pre-trained large language model, analyzes and processes the text information and fusion information through the large language model to predict a requirement of the user, and generates the control information based on the requirement.

In some embodiments, the processor is configured to generate the control information for interacting with the user in a user-initiated mode or a cloud-initiated mode. The cloud-initiated mode indicates that the control unit automatically generate the control information based on detection of the cognitive information through real-time EEG analysis without an input of the user. For example, when specific cognitive information, such as active thinking or memory recall, is detected, a dialogue using AI algorithms is initiated to interpret the EEG signals. The user-initiated mode indicates that the user may use voice commands to engage with the integrated assistance system. For example, the processor may generate the control information when receiving a voice command of asking the target object from the user. In addition, the integrated assistance system utilizes cloud-based ChatGPT to provide real-time assistance, with the initial synthetic voice generated through standard text-to-speech (TTS) technology.

In 550, the processor (e.g., the voice interaction module 460) provides real-time assistance by synthesizing speech based on the control information.

In some embodiments, the processor may synthesize the speech through a text-to-speech (TTS) engine using a voice style transfer technique. In some embodiments, the speech mimics voice of a specific person. For example, the processor may synthesize the speech through the text-to-speech (TTS) engine with a family-style voice, e.g., with a voice of a family member.

To enhance user interaction, the system employs voice style transfer techniques to closely mimic the voice of a specific person, such as a family member. Technologies like VoiceFilter and AutoVC are used to alter the synthetic voice to match the vocal attributes of the specific person, which makes the real-time assistance feel as though it is coming directly from a familiar speech, improving user comfort and engagement. The integration of AI-driven speech synthesis ensures that the speech not only provides assistance or guidance but also feels personal and familiar, significantly improving the overall user experience.

The voice of a specific person may be imitated by integrating speech recognition and natural language processing (NLP) technology, which enhances emotional comfort and user engagement, and helps MCI patients develop a sense of familiarity and trust, which is essential for effective cognitive assistance.

In some embodiments of the present disclosure, EEG signals are integrated with visual sensing and AI-driven algorithms to provide personalized, real-time assistance, which can enhance the quality of life for MCI patients by offering context-aware support through wearable technology and personalized voice interactions that mimic family members, thereby addressing both cognitive and emotional needs.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or collocation of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer-readable program code embodied thereon.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required characteristics of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

What is claimed is:

1. An integrated assistance system, comprising:
a wearable device configured to obtain one or more electroencephalogram (EEG) signals of a user through one or more electrode channels;
an electroencephalogram (EEG) signal sensing module configured to obtain cognitive information by monitoring the one or more EEG signals of the user, the cognitive information including a cognitive load and a memory state, wherein the EEG signal sensing module further includes:
a preprocessing unit configured to preprocess the one or more EEG signals;
an analysis unit configured to determine a cognitive load index (CLI) and an asymmetry index (ASY) by determining a power spectral density (PSD) across a plurality of frequency bands based on the preprocessed EEG signals; and
a cognitive identification unit configured to determine the cognitive load based on the CLI and determine the memory state based on the ASY.

2. The integrated assistance system of claim 1, wherein
the CLI is determined based on a following equation (1):
$$CLI = \frac{{P}_{θ}}{{P}_{α}} , \qquad ( 1 )$$
wherein P_{θ} denotes a power of the one or more electrode channels in a θ band, and
$${P}_{θ} = {P}_{θ}^{A F 7} + {P}_{θ}^{A F 8} + {P}_{θ}^{T P 9} + {P}_{θ}^{TP 10} ;$$
and P_{α} denotes a power of the one or more electrode channels in a α band, and
$${P}_{α} = {P}_{α}^{A F 7} + {P}_{α}^{A F 8} + {P}_{α}^{T P 9} + {P}_{α}^{T P 10} .$$

3. The integrated assistance system of claim 1, wherein
the ASY is determined based on a following equation (2):
$$ASY = \frac{R {H}_{δ} - L {H}_{δ}}{{δ}_{A S Y}} , \qquad ( 2 )$$
wherein RH_{δ} denotes a power of a right hemisphere of the one or more electrode channels in a δ band, and
$$R {H}_{δ} = {P}_{δ}^{A F 8} + {P}_{θ}^{TP 10} ,$$
and LH_{δ} denotes a power of a left hemisphere of the one or more electrode channels in the δ band, and
$$L {H}_{δ} = {P}_{δ}^{A F 7} + {P}_{θ}^{T P 9} ;$$
and δ_{ASY} denotes a standard deviation of an asymmetry index during a baseline period.

4. The integrated assistance system of claim 1, further comprising:
a visual sensing module configured to obtain environment information from an environment of the user; wherein
the data processing unit is further configured to generate the fusion information based on the environment information and the cognitive information.

5. The integrated assistance system of claim 4, wherein the visual sensing module is further configured to determine a target object from the environment of the user using an image recognition model, the image recognition model being a machine learning model.

6. The integrated assistance system of claim 1, further comprising:
a control unit configured to generate control information for interacting with the user based on the fusion information; and
a voice interaction module configured to provide real-time assistance by synthesizing speech based on the control information.

7. The integrated assistance system of claim 6, wherein the voice interaction module further includes a text-to-speech (TTS) engine configured to synthesize the speech using a voice style transfer technique.

8. The integrated assistance system of claim 6, wherein the speech mimics voice of a specific person.

9. The integrated assistance system of claim 6, wherein the control unit is configured to generate the control information for interacting with the user in a user-initiated mode or a cloud-initiated mode, wherein the cloud-initiated mode indicates that the control unit automatically generate the control information based on detection of the cognitive information without an input of the user.

10. An integrated assistance method, comprising:
obtaining one or more electroencephalogram (EEG) signals of a user through one or more electrode channels; and
obtaining cognitive information by monitoring the one or more EEG signals of the user, the cognitive information including a cognitive load and a memory state, wherein the obtaining cognitive information by monitoring the one or more EEG signals of the user includes:
preprocessing the one or more EEG signals;
determining a cognitive load index (CLI) and an asymmetry index (ASY) by determining a power spectral density (PSD) across a plurality of frequency bands based on the preprocessed EEG signals; and
determining the cognitive load based on the CLI and determine the memory state based on the ASY.

11. The integrated assistance method of claim 10, wherein the determining a cognitive load index (CLI) and an asymmetry index (ASY) by determining a power spectral density (PSD) across a plurality of frequency bands based on the preprocessed EEG signals includes:
determining the CLI based on a following equation (1):
$$CLI = \frac{{P}_{θ}}{{P}_{α}} , \qquad ( 1 )$$
wherein P_{θ} denotes a power of the one or more electrode channels in a θ band, and
$${P}_{θ} = {P}_{θ}^{A F 7} + {P}_{θ}^{A F 8} + {P}_{θ}^{T P 9} + {P}_{θ}^{TP 10} ;$$
and P_{α} denotes a power of the one or more electrode channels in a α band, and
$${P}_{α} = {P}_{α}^{A F 7} + {P}_{α}^{A F 8} + {P}_{α}^{T P 9} + {P}_{α}^{T P 10} .$$

12. The integrated assistance method of claim 10, wherein the determining a cognitive load index (CLI) and an asymmetry index (ASY) by determining a power spectral density (PSD) across a plurality of frequency bands based on the preprocessed EEG signals includes:
determining the ASY based on a following equation (2):
$$ASY = \frac{R {H}_{δ} - L {H}_{δ}}{{δ}_{A S Y}} , \qquad ( 2 )$$
wherein RH_{δ} denotes a power of a right hemisphere of the one or more electrode channels in a δ band, and
$$R {H}_{δ} = {P}_{δ}^{A F 8} + {P}_{θ}^{TP 10} ,$$
and LH_{δ} denotes a power of a left hemisphere of the one or more electrode channels in the δ band, and
$${LH}_{δ} = {P}_{δ}^{A F 7} + {P}_{θ}^{T P 9} ;$$
and δ_{ASY} denotes a standard deviation of an asymmetry index during a baseline period.

13. The integrated assistance method of claim 10, further comprising:
obtaining environment information from an environment of the user; and
generating fusion information based on the environment information and the cognitive information.

14. The integrated assistance method of claim 13, wherein the obtaining environment information from an environment of a user includes:
determining a target object from the environment of the user using an image recognition model, the image recognition model being a machine learning model.

15. The integrated assistance method of claim 10, further comprising:
generating control information for interacting with the user based on the fusion information; and
providing real-time assistance by synthesizing speech based on the control information.

16. The integrated assistance method of claim 15, wherein the synthesizing speech based on the control information includes:
synthesizing the speech through a text-to-speech (TTS) engine using a voice style transfer technique.

17. The integrated assistance system of claim 15, wherein the speech mimics voice of a specific person.

18. The integrated assistance method of claim 15, wherein the generating control information for interacting with the user based on the fusion information includes:
generating the control information for interacting with the user in a user-initiated mode or a cloud-initiated mode, wherein the cloud-initiated mode indicates automatically generating the control information based on detection of the cognitive information without an input of the user.

19. A non-transitory computer-readable storage medium storing computer instructions, wherein when reading the computer instructions in the storage medium, a computer executes an integrated assistance method including:
obtaining one or more electroencephalogram (EEG) signals of a user through one or more electrode channels; and
obtaining cognitive information by monitoring the one or more EEG signals of the user, the cognitive information including a cognitive load and a memory state, wherein the obtaining cognitive information by monitoring the one or more EEG signals of the user includes:
preprocessing the one or more EEG signals;
determining a cognitive load index (CLI) and an asymmetry index (ASY) by determining a power spectral density (PSD) across a plurality of frequency bands based on the preprocessed EEG signals; and
determining the cognitive load based on the CLI and determine the memory state based on the ASY.
